(19) United States
(12) Patent Application Publication
(10) Pub. No.: US 2011/0118838 A1
(43) Pub. Date: May 19, 2011

(54) GRAFT PULLEY AND METHODS OF USE

(76) Inventors: George Delli-Santi, Scotts Valley, CA (US); Alan W. Wolf, Kodiak, AK (US)

(21) Appl. No.: 12/751,388

(22) Filed: Mar. 31, 2010

Related U.S. Application Data
(63) Continuation-in-part of application No. 12/619,167, filed on Nov. 16, 2009.

(51) Int. Cl. A61F 2/08

(52) U.S. Cl. 623/13.14; 623/13.13

(57) ABSTRACT

A placement assembly used for positioning and holding a ligament graft within a selected bone tunnel. The assembly includes a graft pulley, a surgical suture, and a graft pulley placement instrument. The graft pulley includes a pulley portion and a bone anchoring portion. The pulley portion is adapted to receive the surgical suture and for surgical suture to be drawn through it, to act as a pulley. The bone anchoring portion comprises a portion adapted to selectively fix the graft pulley within a bone tunnel. The graft instrument may be selectively attached to the graft pulley and includes a handle and elongate body, adapted to insert the graft pulley into the selected bone tunnel. Once the graft pulley is in place, the placement instrument may then be detached and withdrawn leaving the graft pulley firmly fixed within the bone tunnel and can then be used to position a ligament graft.

### CROSS-REFERENCE TO RELATED APPLICATIONS

This application is a continuation-in-part of U.S. application Ser. No. 12/619,167, filed Nov. 16, 2009, and entitled “Graft Pulley and Methods of Use,” hereby incorporated herein by reference.

### FIELD OF THE INVENTION

The present invention relates generally to an apparatus and method for positioning a ligament graft, and more particularly to an implantable graft pulley adapted to firmly fix within a selected bone tunnel and assist the positioning of a graft within said bone tunnel.

### BACKGROUND OF THE INVENTION

One of the most common sports injuries to the knee involves a tearing or detachment of the anterior cruciate (ACL) ligament in the knee. Over the years, surgery to repair this injury has evolved from open reduction surgical procedures on the knee, to less invasive techniques. The goal of the surgery is to attach a graft ligament, between the femur and the tibia. Various grafts may be used, including bone-tendon-bone grafts, soft tissue grafts or an artificial tendon. Whichever graft is used, a typical procedure involves drilling a long tunnel from the anterior proximal end of the tibia, through the distal anterior portion of the femur.

In some procedures a button type fixation device is used, to hold or position one end of the graft in place. The button is typically located external to the knee, close to the bone tunnel exit on the femur and there are numerous disadvantages associated with to this button and the button's location. Firstly, this external location is some distance from the site where the graft may be fixed within the bone tunnel, which makes positioning the graft more difficult and requires additional lengths of sutures to attach the graft to this button. The button also necessitates the extension of the bone tunnel through the femur skin and quadriceps muscle, causing additional trauma to the leg and the increased potential for infections or nerve damage. It is also considered less cosmetically desirable. This external button may also cause interference with the tourniquet used during the surgical procedure.

Several products have more recently been presented that appear to position a graft within a bone tunnel without an incision in the anterior portion of the femur. U.S. Pat. No. 7,381,213, the complete disclosure of which is incorporated herein by reference, describes a radially expanding suture anchor, including a bore formed therein. This system also includes an expander pin to cause the anchor to radially expand within a bone tunnel or hole, into the bone wall to securely attach to bone.

An externally threaded anchor and pulley is described in commonly assigned patent application entitled, “Threaded Pulley Anchor Apparatus and Methods for Use in Surgical Repair of Ligament or Tendon”, U.S. application Ser. No. 11/599,138, filed Nov. 14, 2006, and a bullet-shaped anchor is described in commonly assigned patent application entitled, “External Bullet Anchor Apparatus and Method for Use in Surgical Repair of Ligament of Tendon”, U.S. application Ser. No. 11/595,353, filed Nov. 9, 2006, the complete disclosures of which are incorporated herein by reference.

### SUMMARY OF THE INVENTION

The present disclosure presents an improved placement assembly for positioning a ligament graft within a selected bone tunnel. The assembly includes a surgical suture which may be assembled with a graft pulley, and a graft pulley placement instrument removably connected with said graft pulley. The graft pulley has a pulley portion and a bone anchoring portion. The pulley portion includes an aperture or channel sized to receive the surgical suture, so that the surgical suture may be drawn through the aperture, to act as a pulley. The bone anchoring portion includes at least one flexible wing that is adapted to selectively fix the graft pulley within a bone tunnel. The placement instrument is adapted to place the graft pulley within the bone tunnel and also to assist in fixing it within the bone tunnel and the instrument includes a handle and elongate body. Once the graft pulley is in place, the instrument may then be detached from the graft pulley and withdrawn from the bone tunnel.

In another aspect a graft pulley is disclosed adapted for positioning a ligament graft within a selected bone tunnel. The graft pulley includes a pulley portion having an aperture or channel sized to receive surgical suture and a bone anchoring portion having at least one flexible wing, adapted to selectively fix the graft pulley within a bone tunnel.

In yet another aspect a graft pulley for positioning a ligament graft within a bone tunnel is disclosed, including a pulley portion having an aperture or channel sized to receive surgical suture and a bone anchoring portion.

In yet another aspect a method of performing a medical procedure on a body is disclosed. The method includes drilling a bone tunnel and then inserting a graft pulley into the bone tunnel. The graft pulley includes a pulley portion and a bone anchoring portion. The pulley portion has an aperture sized to receive a surgical suture and the bone anchoring portion has at least one flexible wing adapted to fix the graft anchor within a bone tunnel. At least one wing is then flexed in a second direction, so as to slide the graft pulley into the bone tunnel in a first direction, and once the graft pulley is in place the graft pulley is retracted in the second direction, to firmly fix the graft pulley within the bone tunnel.

A further aspect of a graft pulley is disclosed for positioning a ligament graft within a selected bone tunnel. The bone tunnel has a blind end that is spaced substantially from the capsule of the bone that the tunnel is disposed. The graft pulley includes a pulley portion with an aperture that is sized to receive at least one surgical suture, as well as a bone anchoring portion, which may be selectively advanced into the blind end of the selected bone tunnel, so that a portion of the bone anchoring portion is disposed within the bone tunnel blind end to secure the graft pulley in position. The graft pulley is sized so as to not engage or violate the bone tunnel walls during insertion. The bone anchoring portion may include a self tapping tip that may be advanced or screwed beyond the bone tunnel blind end to fix the graft pulley within the bone tunnel blind end. A pilot hole may be present within the bone tunnel blind end to help guide the self tapping tip.

A further aspect of a tissue pulley is disclosed for positioning a target tissue proximal to a selected bone tunnel. The bone tunnel has a blind end which may be substantially spaced from a bone capsule. The tissue pulley includes a pulley portion with an aperture that is sized to receive at least one surgical suture, as well as a bone anchoring portion. The bone anchoring portion comprises a threaded surface that may be selectively advanced beyond the bone tunnel blind end so as to cut a pathway towards the bone capsule and is advanced so that a portion of the bone anchoring portion is disposed within the blind tunnel blind end so to fixedly secure the pulley with the bone. The graft pulley may be sized to as to not violate any bone tunnel walls during operation.

In a further aspect, a method of performing a medical procedure on a patient's knee is disclosed. The method includes the steps of drilling a bone tunnel, so that the bone tunnel has a blind end, which is spaced substantially from a femur capsule distal end. A graft pulley is then placed into the bone tunnel, the graft pulley being sized so as to not violate the bone tunnel walls during placement. The graft pulley includes a pulley portion and a bone anchoring portion, wherein the pulley portion has an aperture sized to receive at least one surgical suture and the bone anchoring portion may be inserted up to the bone tunnel blind end. The bone anchoring portion is then positioned up against the bone tunnel blind end followed by the step of rotating the graft pulley so as to advance the bone anchoring portion into the bone tunnel blind end. This bone anchoring portion may have a self tapping tip and inserting the bone anchoring portion may then include screwing this self tapping tip into the bone tunnel blind end so that a portion of the anchoring portion may be disposed within the bone tunnel blind end. A ligament graft may then be attached to the surgical suture and at least one suture may then be drawn through the anchor pulley aperture to position the ligament graft to a desired position within the bone tunnel before the graft is fixed to the bone tunnel.

In a further aspect, a method of performing a medical procedure on a body is disclosed. The method includes the steps of drilling a bone tunnel, so that the bone tunnel has a blind end, spaced substantially from a bone capsule, followed by threading a surgical suture though a ligament, wherein the suture has two suture ends. The two suture ends are then passed through a graft pulley portion, which is sized to receive at least two surgical sutures. The graft pulley is then inserted into the bone tunnel using a pulley placement instrument, the graft pulley comprising the pulley portion with the suture ends assembled, together with a bone anchoring portion. The bone anchoring portion has a threaded self tapping tip. The graft pulley is then positioned up against the bone tunnel blind end and then rotated and advanced, using the placement instrument, into the tunnel blind end, sufficient to fixedly secure the graft pulley with the bone blind end, so that a portion of the bone anchoring portion is disposed within the tunnel blind end. The pulley placement instrument may then be removed from the tunnel and the two suture ends drawn out of the tunnel to position the ligament graft in a desired position within the bone tunnel. One suture end may then be withdrawn from pulley aperture and bone tunnel and the ligament may then be fixed to bone tunnel.

The present disclosure includes a number of important technical advantages. One technical advantage is that the pulley may be precisely, quickly and easily slid into place. Another advantage is that the pulley may not cause any significant trauma to the bone tunnel wall during insertion as well as during fixation. Another advantage is that this invention does not require an incision in the anterior femur, reducing the trauma to the femur and likelihood of an additional wound site infection, unsightly markings of the incision site and interference with tourniquets or equipment used during surgery. Another advantage is that the graft pulley may be placed closer to where the graft may be anchored, potentially reducing the length of sutures required for the procedure, and thereby reducing the complexity of the procedure. Another advantage is that the graft may be easily positioned within the bone tunnel without extra placement equipment. Additional advantages will be apparent to those of skill in the art and from the figures, description and claims provided herein.

### BRIEF DESCRIPTION OF THE DRAWINGS

The invention may best be understood by reference to the following description taken in conjunction with the accompanying drawings in which:

FIG. 1A shows a placement assembly according to the teachings of the present disclosure;

FIG. 1B shows a cross section of a placement assembly according to the teachings of the present disclosure;

FIG. 2 shows a graft pulley according to the teachings of the present disclosure;

FIG. 3 shows a partial view of a placement assembly, disassembled to show the suture, according to the teachings of the present disclosure;

FIG. 4A shows a placement assembly before insertion into a bone tunnel according to the teachings of the present disclosure;

FIG. 4B shows a placement assembly during insertion into a bone tunnel according to the teachings of the present disclosure;

FIGS. 5A, B and C are representations of a graft pulley in place and a ligament graft attached with alternative embodiments of suture routing;

FIGS. 6 and 6B show flow diagrams of methods of using the present invention in a medical procedure, according to the teachings of the present disclosure;

FIGS. 7A and 7B show longitudinal cross sectional views of alternative graft pulley embodiments;

FIG. 8 shows an alternative embodiment of a graft pulley;

FIG. 9 shows a graft pulley within a bone tunnel blind end according to at least certain embodiments of the present disclosure; and

FIGS. 10 and 11 show a flow diagram of a medical procedure for using a graft pulley disposed within the blind end of a bone tunnel, according to at least certain embodiments of the present invention.

### DETAILED DESCRIPTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

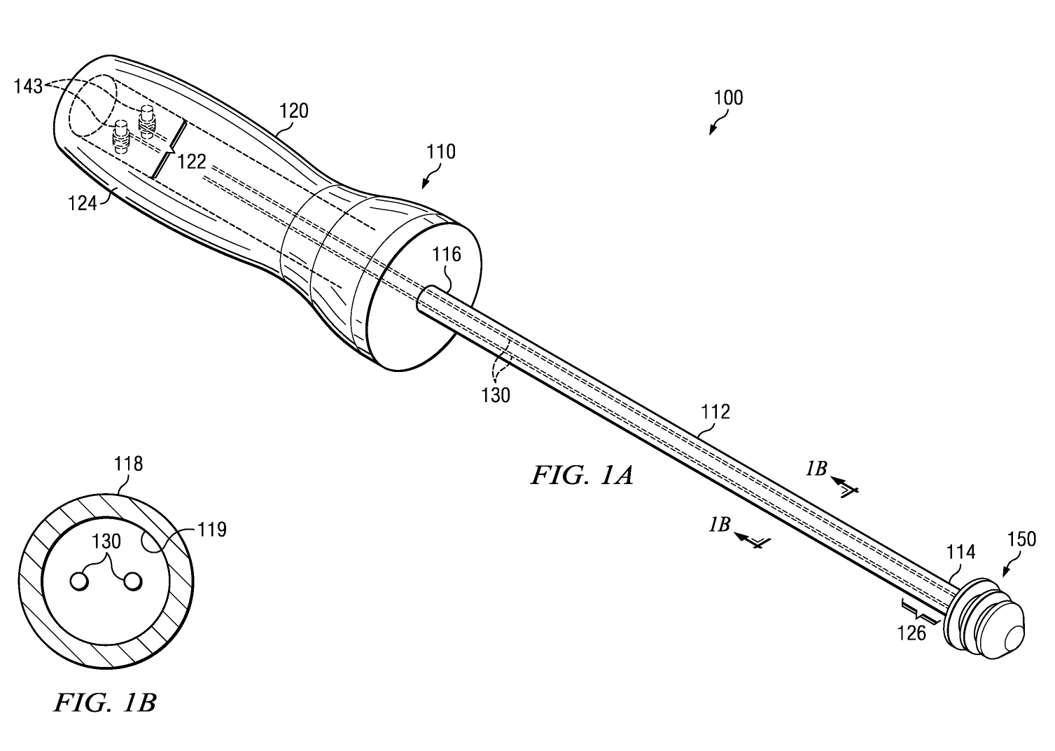

FIG. 1A shows a placement assembly 100 according to the teachings of the present disclosure. Assembly 100 generally includes at least one graft pulley placement instrument 110, at least one surgical suture 130 (shown in phantom) and at least one graft pulley 150.

Instrument 110 includes a handle 120 and an elongate body 112; body 112 may be tubular. Elongate body 112 includes a distal end 114 and proximal end 116 to which instrument handle 120 is preferably connected. Handle 120 generally facilitates manipulation of instrument 110. In the present embodiment a suture securing portion 122 is formed within handle 120 or may alternatively be formed on handle surface 124. Securing portion 122 is preferably adapted to temporarily secure suture 130 until graft pulley 150 has been inserted. Suture securing portion 122 may include at least one post or tab 143 that suture 130 wraps around. In alternative embodiments securing portion 122 may include grooves or hooks to secure suture 130. Handle 120 may include a clam shell type recess or slot (not expressly shown) to access the suture 130 once pulley 150 is in place. The present embodiment shows a barrel type handle 120; however the present invention may be employed with any suitable handle shape of configuration such as T-grip type handle.

Instrument elongate body 112 may generally include a smooth outer surface 118 and an inner luminal surface 119 as shown in FIG. 1B. Elongate body distal end 114 may also include a connecting portion 126, adapted to removably connect with graft pulley 150. As shown in FIGS. 1A and 1B, suture 130 may extend from graft pulley 150 along inner lumen 119 to instrument handle 120 and instrument handle securing portion 122. Suture 130 may be contained within elongate body 112. In alternative embodiments not shown here, suture 130 may extend along a groove positioned longitudinally along elongate body outer surface 118 or juxtaposition upon the outside surface 118 of elongate body. Alternative embodiments with the suture 130 on the outside of the elongate body may be preferable should the surgeon wish to pre-attach the ligament before pulley 150 insertion.

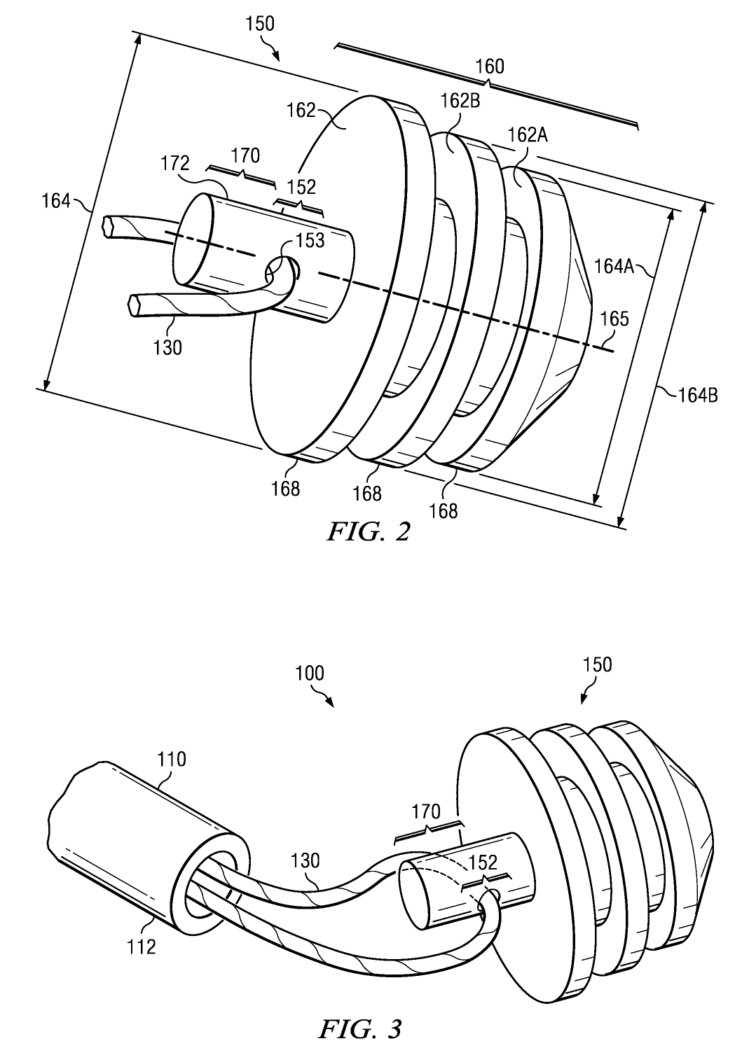

Graft pulley 150 is shown in more detail in FIG. 2. Graft pulley 150 generally includes a pulley portion 152, a bone anchoring portion 160 and an instrument attachment portion 170. Graft pulley 150 is preferably constructed from an implant grade material and portions of graft pulley 150 may also be constructed from a radiopaque material to aid in visualization using imaging techniques such as fluoroscopy or radiography. Bone anchoring portion 160 may comprise at least one flexible wing 162 adapted to secure graft pulley 150 within a bone tunnel. In the present embodiment wing 162 is a substantially flat, circular disc. In alternative embodiments, wing 162 may be non-cylindrical and/or non-circular in shape and may be any shape suitable for anchoring techniques described herein. In alternative embodiments, wing 162 may have a uniform thickness, as shown in the present embodiment, or may have a variable thickness. Each wing 162 has a non-flexed cross sectional dimension 164, measured when wing 162 is in a neutral relaxed position and each wing 162, 162A and 162B may have a different cross sectional dimension 164, 164A and 164B from any other wing, to account for variation in bone tunnel size or shape and to facilitate insertion into a bone tunnel. Alternatively, more or fewer flexible wings 142 may be provided. In some alternate embodiments, flexible wings 142 may have a substantially uniform cross sectional dimension 164. Three flexible wings 162, 162A and 162B are shown in FIG. 2, each with differing cross sectional dimensions 164. As shown in FIG. 2, each wing 162, 162A and 162B is progressively smaller cross sectional dimension 164, 164A and 164B. As shown, a first wing 162A includes a cross sectional dimension 164A and a second wing 162B includes a cross sectional dimension 164B. In the present embodiment first cross sectional dimension 164A is smaller than second cross sectional dimension 164B. In alternative embodiments wing 162 may not form a continuous disc or other shape, but may include slots or gaps in wing 162 (not expressly shown). In the present embodiment wing 162 is shown positioned approximately perpendicular to longitudinal axis 165.

Wing 162 may be formed so as to preferably flex during insertion into a bone tunnel and resist flex and wedge into the bone tunnel if pulley 150 is pulled in a direction approximately opposing the direction of insertion. This flexing occurs as a result of inserting pulley 150 into the tunnel, as they inherently bend downstream as a result of insertion, so as to fit within tunnel. In the embodiment shown in FIG. 2, at least one wing cross sectional dimension 164 is designed to be larger than the intended bone tunnel diameter (described in a later figure). Wings 162, 162A and 162B may preferably be constructed from a flexible material such as high density polyethylene.

In some embodiments, wing edges 168 may be formed to facilitate fixation of pulley 150. For example, wing edges 168 may be formed with a surface texture (not expressly shown) and/or a high friction coating may be disposed on wing edge 168 to increase friction between wing edge 168 and the bone tunnel wall and thereby increase the relative fixation strength or so-called pull-out strength of pulley 150 after implantation. However, the expected load on pulley 150 during ligament insertion and positioning may be relatively small as pulley 150 may be used primarily for positioning of a ligament within the bone tunnel and other surgical instruments may assist in positioning. Following the positioning of the ligament, the ligament may be held in tension, thereby exerting a force on pulley 150, as a suitable fixation device, such as a cannulated screw or expandable device, is inserted within the bone tunnel and secure the ligament therein.

Pulley portion 152 includes at least one opening or aperture 153 sized to receive surgical suture 130. Pulley portion aperture 153 is sized so as to allow standard sized surgical suture 130 to easily slide through said aperture 153 and may be oriented approximately perpendicular to longitudinal axis 165. Pulley portion 152 may be made from substantially low friction materials such as polyolefin to allow suture 130 to easily slide through aperture 153. In alternative embodiments, not shown here, aperture may be a channel or tunnel that extends into pulley 150 and pulley portion 152 may be fully or partially recessed within bone anchoring portion 160.

Graft pulley instrument attachment portion 170 is generally adapted to removably attach to an instrument (not shown here) used to insert graft pulley 150 within a bone tunnel. Shown here, graft attachment portion 170 is a shaft 172. An instrument with a hollow lumen (not shown here) may then removably slide over said shaft 172 to manipulate pulley 150 into a bone tunnel. An instrument may then slide off shaft 172 to disengage. Shaft 172 may be substantially circular in shape. Alternate embodiments may include a substantially non circular shaft 172, which may aid in pulley 150 orientation or rotation if needed. Shaft 172 may preferably be sized to insert the pulley 150 into the tunnel but easily slide off once retracted. In alternative embodiment, suture 130 may also cause a frictional or interference fit between the shaft 172, suture 130 and the instrument lumen. In alternative embodiments the shaft 172 may be adapted to selectively disengage from pulley 150, via release mechanisms or friction fits, not shown here.

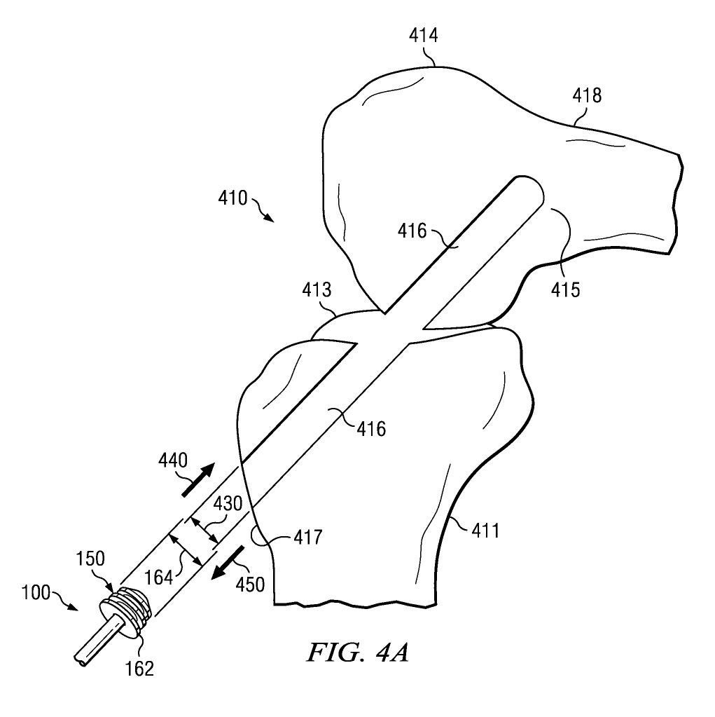

FIG. 3 shows a partial view of a placement assembly 100, shown with instrument 110 disconnected from anchor 150. Assembly 100 generally includes a graft pulley 150 having a pulley portion 152 and instrument attachment portion 170, surgical suture 130 and placement instrument 110. Shown in FIG. 3 is an embodiment with suture 130 assembled within instrument elongate body 112. Suture 130 is shown assembled with pulley portion 152. Instrument attachment portion 170 is adapted to removably engage with distal end 114 of elongate body 112.

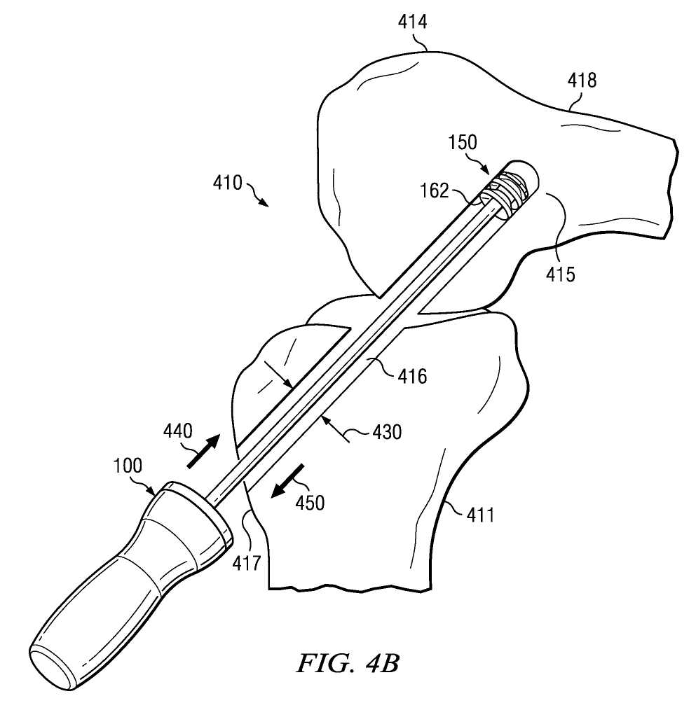

FIG. 4A shows a placement assembly 100 ready to be inserted into a patient's knee 410. Knee 410 is shown with tibia 411 approximately ninety degrees with respect to femur 414. A bone tunnel 416 has been formed in an upper anterior portion 417 of the tibia 411, extending into a distal end 415 of femur 414 through knee capsule 413. In the present embodiment, the distal end of the femur capsule 418 may not be punctured, that is, bone tunnel 416 extends only partially into femur 414. In some embodiments, bone tunnel 416 may include a guide or pilot hole that does extend through the end of femur capsule 418. Bone tunnel 416 has a bone tunnel diameter 430. In alternate embodiments, bone tunnel diameter may not be constant along the length the of bone tunnel 416.

Graft pulley 150 is shown with at least one wing 162 that has a non-flexed cross sectional dimension 164 that is larger than bone tunnel diameter 430. During the insertion of assembly 100 in first direction 440, at least one wing 162 may preferably flex in second direction 450 to allow for slideable insertion of assembly down bone tunnel. Bone tunnel diameters 430 may vary in size depending on the tool a surgeon chooses to drill said tunnel. Some exemplary diameters 430 vary from 7 mm to 12 mm and therefore a variety of wing cross sectional dimensions 164 may also be provided. Wing cross sectional dimensions 164 may be approximately between 1 mm and 4 mm greater than the bone tunnel diameter 430 and more preferably be approximately 1-2 mm greater than the respective bone tunnel diameter 430. Therefore, for example, for a tunnel diameter 430 that is approximately 10 mm, a pulley cross sectional dimension 164 may preferably be no larger than approximately 12 mm at any point on at least one wing 162. This cross sectional dimension 164 may depend on material properties such as elastic modulus and shape, thickness and size of the wing 162.

Placement assembly 100 is then preferably inserted into bone tunnel 416 in first direction 440 as shown in FIG. 4B and pulley 150 is shown with at least one wing 162 that is flexed to facilitate insertion. As pulley 150 enter bone tunnel 416, wing 162 preferably flexes in second direction 450 to conform to tunnel diameter 430, which is smaller than wing non flexed cross sectional dimension 164. Thereafter, wing 162 will resist any attempt to withdraw or retract pulley 150 in second direction 450. This resistance provided by wing 162 is preferable sufficient to position a ligament graft as described below. In a preferred embodiment, wing 162 will preferably not flip or flex to first direction 440 and will essentially wedge graft pulley 150 in place within the tunnel. However, if pulley 150 is not considered deep enough in the tunnel 416 at any time, pulley 150 may be easily repositioned further along the first direction, into the tunnel. Instrument may be re-engaged in order to reposition pulley 150, if instrument has been disengaged. In the present embodiment pulley 150 will preferably not burrow or dig into bone tunnel 416 during use.

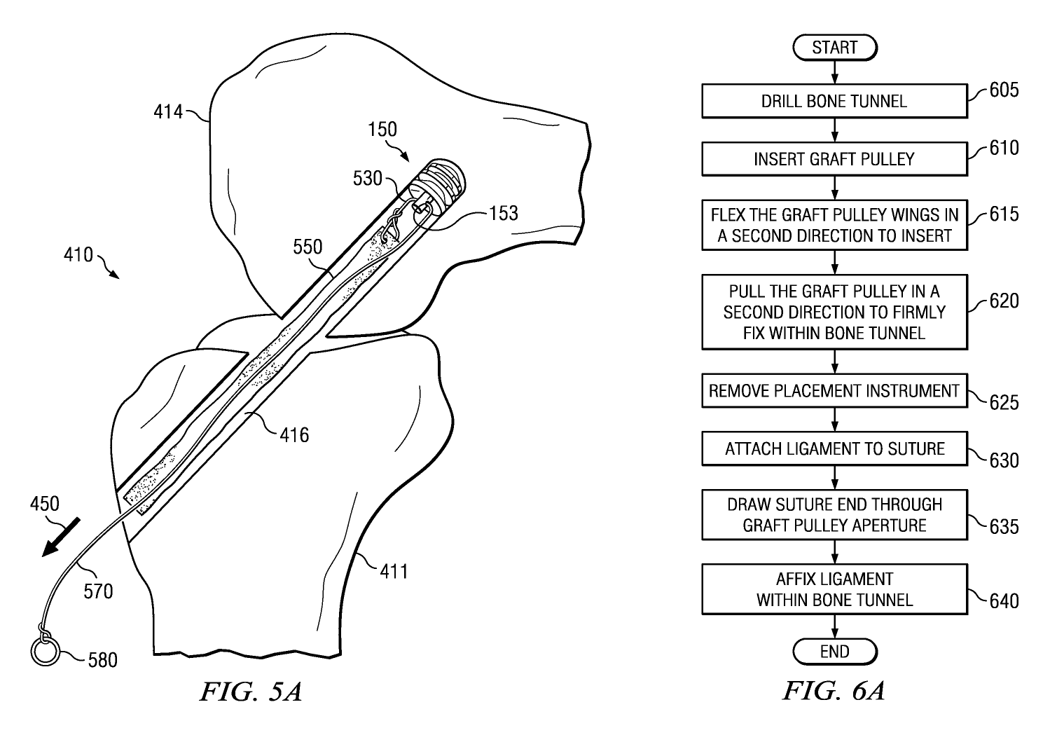

FIG. 5A shows a ligament graft 550 being positioned within bone tunnel 416. Similar to FIG. 4, knee 410 is shown with tibia 411 and femur 414 in a ninety degree position. Graft pulley 150 is shown firmly fixed within bone tunnel 416. Graft 550 is shown attached to a suture first end 530. A suture needle (not shown) may be used to attach ligament 550 to suture 530. A suture second end 570 may then be pulled in second direction 450 out of tunnel 416, to position graft 550 in place within tunnel 416. Suture second end 570 may also include a stop button or stop ring 580 adapted to prevent suture second end 570 from travelling into bone tunnel 416 and potentially through pulley aperture 153. Once ligament 550 is in place ligament, suture end 570 may be cut as close to the pulley as possible. Alternately suture may left within the tunnel 416 and may be made of a suitably absorbable material. Ligament 550 may be further anchored into place using traditional anchors and instruments for ligament repair as are well known to those of skill in the art.

FIG. 5B shows a ligament graft 550 being positioned within bone tunnel 416 using an alternative suture routing. This alternate routing may allow the surgeon the ability to remove the suture 530 from the tunnel 416, if desired. Similar to FIG. 4, knee 410 is shown with tibia 411 and femur 414 in a ninety degree position. Graft pulley 150 is shown firmly fixed within bone tunnel 416. Graft 550 is shown with suture 530 looped through ligament 550 and then two suture ends 531 and 571 routed through pulley aperture 153 together, in the same direction. A suture needle (not shown) may be used to attach ligament 550 to suture 530, and for this alternate suture routing, suture may be attached to ligament 550 and routed through pulley 150, before placement of pulley 150 within bone tunnel 416. Alternatively, a suture shuttle (not expressly shown) may be integral to the insertion instrument to route the suture according to FIG. 5B. Suture ends 531 and 571 may then be pulled, in tandem, in second direction 450 out of tunnel 416, to position graft 550 in place within tunnel 416. At least one of suture ends 531 or 571, may also include a stop button or stop ring 580, adapted to prevent suture second end 571 from travelling into bone tunnel 416 and potentially inadvertently through pulley aperture 153. Once ligament 550 is in place, suture end 531 may then be pulled in second direction 450 so as to withdraw suture 530 completely from pulley 150 and ligament 550 and out of tunnel 416. Ligament 550 may then be further anchored into place using traditional anchors and instruments for ligament repair as are known to those of skill in the art.

FIG. 5C shows a ligament graft 550 being positioned within bone tunnel 416 using an alternative routing of a suture, similar in spirit to FIG. 5B. This alternate routing may allow the surgeon the ability to remove the suture 530 from the tunnel 416. Similar to FIG. 4, knee 410 is shown with tibia 411 and femur 414 in a ninety degree position. Graft pulley 150 is shown firmly fixed within bone tunnel 416. Graft 550 is shown with suture 530 attached to ligament 550 and then two suture ends 531 and 571 are routed through pulley aperture 153. The two suture ends 531 and 571 are routed through the graft pulley 150 in opposing directions. A suture needle (not shown) may be used to attach ligament 550 to suture 530, and for this alternate suture routing, suture may need to be attached to ligament 550 and pulley 150, before placement of pulley 150. Alternatively, a suture shuttle (not shown) may be integral to the insertion instrument to route the suture according to FIG. 5B. Suture ends 531 and 571 may then be pulled, in tandem, in second direction 450 out of tunnel 416, to position graft 550 in place within tunnel 416. At least one of suture ends 531 or 571, may also include a stop button or stop ring 580 adapted to prevent suture second end 570 from travelling into bone tunnel 416 and potentially inadvertently through pulley aperture 153. Once ligament 550 is in place ligament, suture end 531 may then be pulled in second direction 450 so as to withdraw suture 530 from pulley 150 and ligament 550, and subsequently out of tunnel 416. Ligament may then be further anchored into place with using traditional anchors and instruments for ligament repair as are known to those of skill in the art.

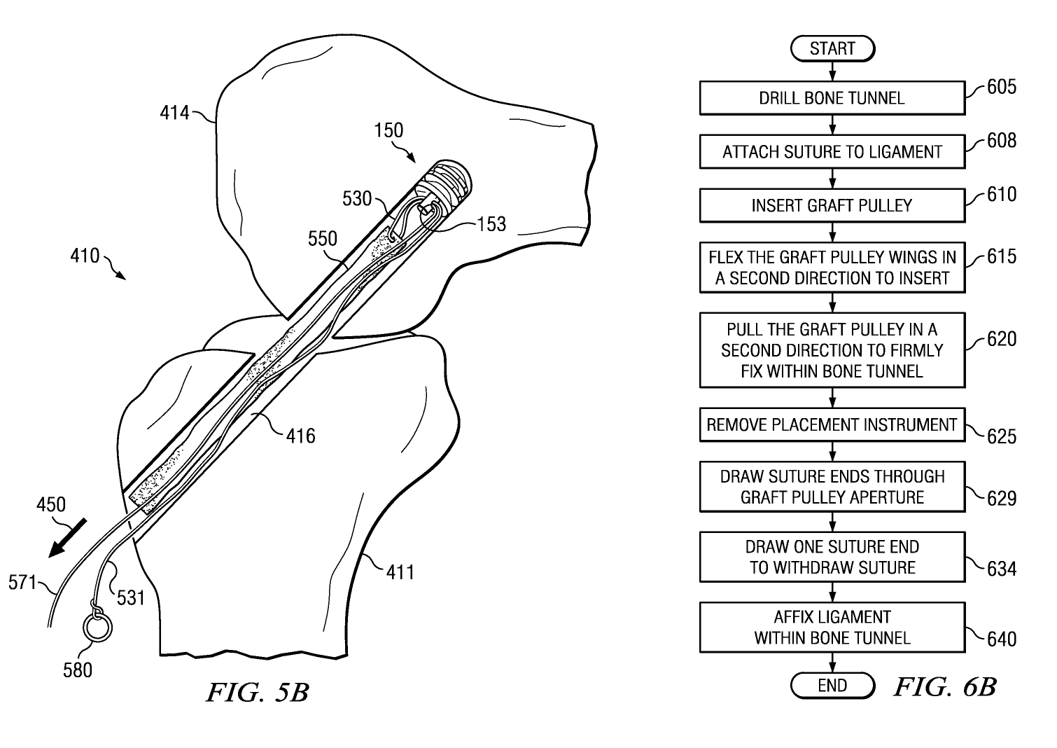

A method of performing a portion of a medical procedure using a graft pulley is shown in FIG. 6A. A knee joint is first positioned at a ninety degree angle and a bone tunnel is drilled 605 from an upper anterior portion of a tibia through a knee capsule and partially within a distal end of a femur without penetration into a lateral femoral cortex. A graft pulley is then inserted 610, using a placement instrument. Graft pulley includes a pulley portion sized to receive surgical suture and a bone anchoring portion; the anchoring portion includes one or more flexible wings adapted to fix the graft pulley within the bone tunnel. The wings may be flexed 615 in a second direction as the graft pulley is inserted into the bone tunnel in the first direction. The graft pulley may then be pulled in a second direction 620 to firmly fix graft pulley within bone tunnel. Placement instrument may then be detached and withdrawn 625, leaving graft pulley in place.

Thereafter a ligament graft may be provided and attached 630 to surgical suture. Suture end may then be drawn 635 through graft pulley aperture in order to position graft within a bone tunnel. If the pulley is not deemed far enough along the tunnel, at any time, an additional step may be added, further inserting the pulley into the bone tunnel before the step of detaching the placement instrument. Alternatively, the placement instrument may be re-engaged.

The ligament may then be fixed in position within the bone tunnel 640. Fixing the ligament more permanently to the tunnel wall is achieved using a large variety of bone anchors that are well known to one skilled in the art. The suture may be left within the tunnel, or may be cut during the step of fixing the ligament in place, leaving only a portion of suture in-situ. Alternatively, the surgeon may chose to cut the suture before the step of fixing the ligament in position.

An alternate method of performing a portion of a medical procedure using a graft pulley is shown in FIG. 6B. A knee joint is first positioned at a ninety degree angle and a bone tunnel is drilled 605 from an upper anterior portion of a tibia through a knee capsule and partially within a distal end of a femur without penetration into a lateral femoral cortex. A suture may then be attached 608 to a ligament to preassemble the suture and ligament before pulley insertion. A suture may also be inserted through a pulley portion of a graft pulley sized to receive surgical suture. A graft pulley is then inserted 610, using a placement instrument. Graft pulley includes a pulley portion and a bone anchoring portion; the anchoring portion includes one or more flexible wings adapted to fix the graft pulley within the bone tunnel. The wings may be flexed 615 in a second direction as the graft pulley is inserted into the bone tunnel in the first direction. The graft pulley may then be pulled in a second direction 620 to firmly fix graft pulley within bone tunnel. Placement instrument may then be detached and withdrawn 625, leaving graft pulley in place.

Both suture ends may then be drawn 629 through graft pulley aperture, in order to position graft within a bone tunnel. Once ligament is in position, one suture end may then be drawn 634 so as to withdraw the suture from the tunnel. The ligament may then be fixed in position within the bone tunnel 640.

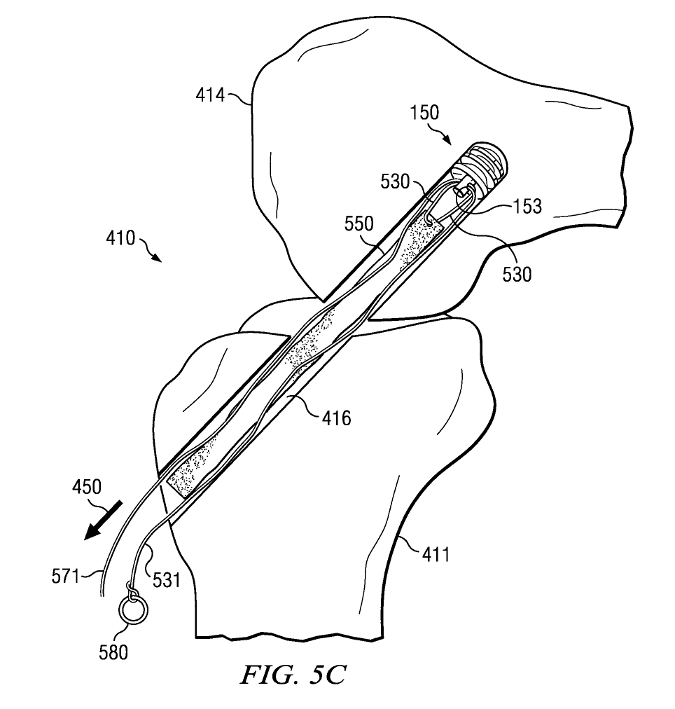

FIG. 7A shows a longitudinal cross section of an alternative embodiment of a graft pulley 700. Graft pulley 700 is similar in spirit to the previous pulley described and includes a pulley portion 702, attachment portion 704, longitudinal axis 705 and at least one wing 720. At least one wing 720 is formed at an angle 725 to longitudinal axis 705. Angle 725 may improve the ease of pulley 700 insertion into a bone tunnel. Wing non-flexed outer cross sectional dimension 730 may be substantially larger than an intended bone tunnel diameter to make graft pulley 700 wedge within the tunnel when a retraction force is applied. Wing 720 may be circular or non-circular.

FIG. 7B shows a longitudinal cross section of an alternative embodiment of a graft pulley 750. Pulley 750 is similar in spirit to previous pulley described and includes a pulley portion 752, attachment portion 754, longitudinal axis 755 and at least one wing 770. At least one wing 770 is formed in the shape of a hook 775. Hook 775 may improve the ease of pulley 750 insertion into a bone tunnel and provide for improved affixation strength. Wing non flexed outer cross sectional dimension 780 may be larger than an intended bone tunnel diameter to make pulley 750 wedge within a bone tunnel. Wing 770 may be circular or non-circular.

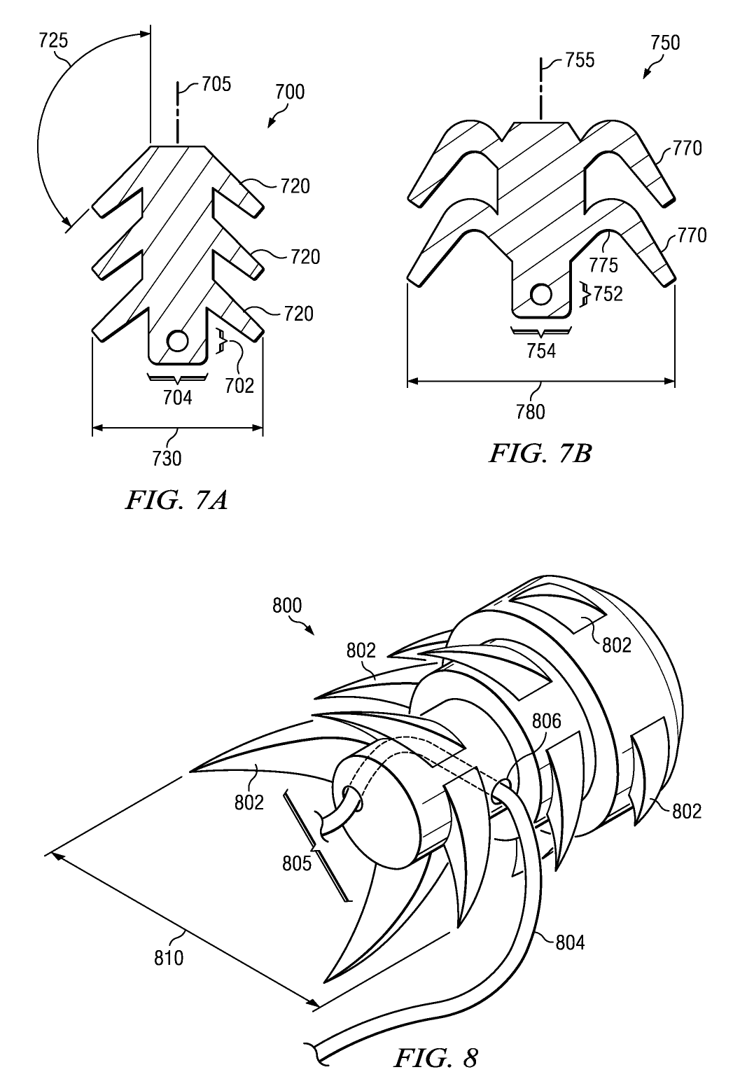

FIG. 8 shows a view of an alternative embodiment of a graft pulley 800. Pulley is similar in spirit to pulleys previously described and includes at least one radially extending flexible fin or hook 802 formed to firmly fix the pulley 800 within a bone tunnel (not shown here). A series of hooks or fins 802 may be formed in rows (as shown) or fins 802 may be arranged in a helix or randomly disposed along pulley 800. Similar to previous figures, the non-flexed cross sectional dimension 810 may preferably be selected to be larger than a respective bone tunnel diameter (not shown here). At least one fin 802 may flex upon pulley 800 insertion into a bone tunnel in a first direction and then resist flex when pulled in a second direction out of said bone tunnel, effectively affixing pulley 800 within bone tunnel. This pulley 800 embodiment may adapt better to uneven bone tunnel shapes.

Pulley 800 also shows a pulley portion 805 including a suture aperture 806 in a channel shape, located inside pulley 800. Suture 804 is shown threaded along suture channel 806 and is similar in spirit to previous pulley portions described. This embodiment may show improved durability over previous embodiments described as it is internal to the body of the pulley 800. Alternate embodiments (not shown here) may include a pulley with an anchoring portion comprising at least two fins or hooks, legs or wings. These fins etc. may have a cross sectional dimension smaller than that of a bone tunnel and with heat or mechanical activation, at least one fin may recover to a position that increases the cross sectional dimension to a size larger or equal to a bone tunnel diameter, thus wedging the pulley within the bone tunnel.

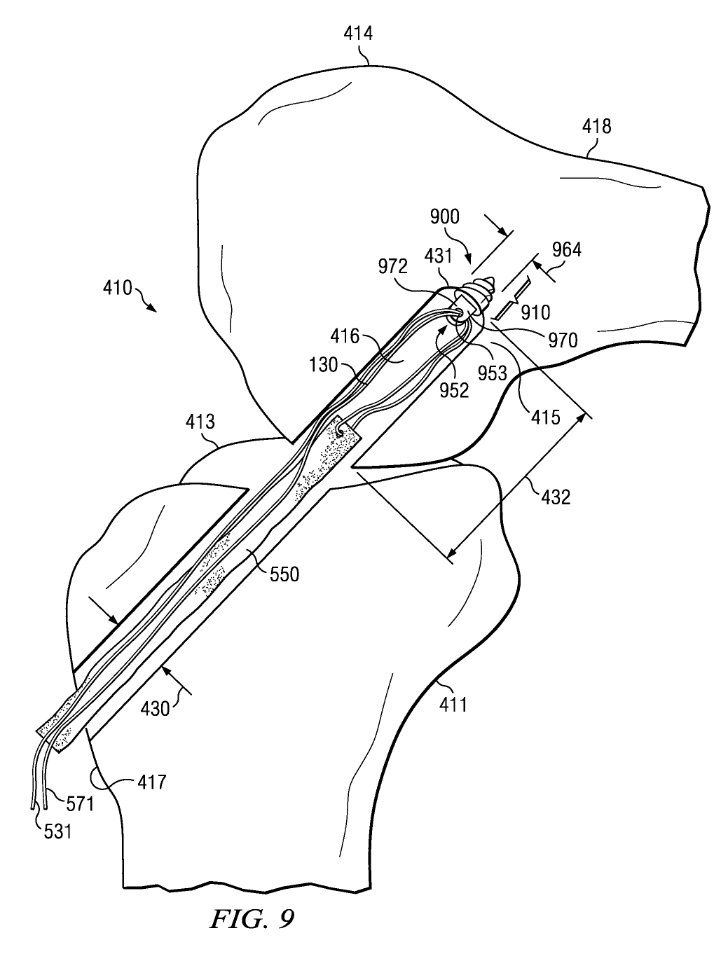

FIG. 9 shows a graft pulley 900 inserted into a patient's knee 410. Knee 410 is shown with tibia 411 approximately ninety degrees with respect to femur 414, similar to that described in earlier figures. A bone tunnel 416 has been formed in an upper anterior portion 417 of the tibia 411, extending partially into a distal end 415 of femur 414 through knee capsule 413. In the present embodiment, the distal end of the femur capsule 418 may not be punctured, that is, bone tunnel 416 extends only partially into femur 414 and blind end 431 is spaced substantially from femur distal end 418. Furthermore, using graft pulley 900 may allow tunnel 416 to extend minimally into femur and be spaced further from femur capsule distal end 418 compared with alternative anchor concepts, so that graft pulley 900 may cause less trauma and potential weakening to the bone. Extension 432 may be shorter or reduced, compared with alternative anchor concepts that engage the tunnel walls such as those described earlier in this disclosure, for example. In some embodiments, bone tunnel 416 may include a guide or pilot hole (not shown here) that extends at least partially from the tunnel blind end 431. It may not necessarily extend through the end of femur capsule 418.

Graft pulley 900 is shown with a cross sectional dimension 964 that may preferably be smaller than bone tunnel diameter or cross sectional dimension 430. In some embodiments, bone tunnel diameter 430 may not necessarily be constant along the length of bone tunnel 416; however the tunnel wall may preferably not be compromised, engaged or violated substantially during insertion, as the pulley cross sectional dimension 964 is smaller than tunnel dimension 430 along the tunnel length. Contact may naturally occur during insertion, but minimal damage or impinging may occur. Bone tunnel diameters 430 may vary in size depending on the tool a surgeon chooses to drill said tunnel. Some exemplary dimensions 430 vary from 7 mm to 12 mm and therefore a variety of graft pulley cross sectional dimensions 964 may also be provided. Graft cross section dimensions 964 may be approximately between 0.5 mm and 4 mm smaller than the bone tunnel diameter 430 and more preferably is approximately 1-2 mm smaller than the respective bone tunnel diameter 430. Therefore, for example, for a tunnel diameter 430 that is approximately 10 mm, a pulley cross sectional dimension 964 may preferably be no larger than approximately 9 mm at any point.

Graft pulley 900 generally includes a pulley portion 952, a bone anchoring portion 910 and an instrument attachment portion 970. Graft pulley 900 is preferably constructed from an implant grade material and portions of graft pulley 900 may also be constructed from a radiopaque material to aid in visualization using imaging techniques such as fluoroscopy or radiography. Pulley portion 952 includes at least one opening or aperture 953 sized to receive at least one surgical suture 130 and is similar in spirit to the pulley portion embodiments described earlier. Two suture ends 571 and 531 are shown passed through pulley portion aperture 953, for an exemplary suture routing through said aperture 953, similar in spirit to FIG. 5B. Alternative suture routing embodiments are also described previously and aperture 953 may be sized to allow any number, including one standard sized surgical suture 130 to easily slide through said aperture 953 depending on the preferred suture routing method. Aperture 953 may be oriented approximately perpendicular to the graft pulley longitudinal axis. Aperture 953 may have rounded edges to allow the easy movement of suture 130 through aperture 953. Pulley portion 952 may be made from substantially low friction materials, such as polyolefin, to allow suture 130 to easily slide through aperture 953. In alternative embodiments, not shown here, aperture may be a channel or tunnel that extends into pulley 900 and pulley portion 952 may be fully or partially recessed within bone anchoring portion 910.

Graft pulley instrument attachment portion 970 is generally operable to be removably engaged with an instrument (not shown here) used to insert graft pulley 900 within a bone tunnel and subsequently fix the graft pulley 900 with the tunnel blind end 431. Shown here, graft pulley instrument attachment portion 970 is a shaft 972. An instrument with a hollow lumen (not shown here) may then removably slide over said shaft 972 to manipulate pulley 900 into a bone tunnel. The instrument may then slide off shaft 972 to disengage from shaft 972. Shaft 972 may be substantially circular in shape. Alternate embodiments may include a substantially non circular shaft, such as a hexagonal shape, rectangular shape shaft, or a shape with at least one flat side, which may aid in achieving a desired orientation, rotation and fixation with regard to pulley 900. Shaft 972 may preferably be sized to insert the pulley 900 into the tunnel but also operable to easily slide off once retracted. In alternative embodiments, suture 130 may also be part of a frictional or interference fit between the shaft 972, suture 130 and the instrument lumen. In further alternative embodiments, the shaft 972 may be adapted to selectively disengage from pulley 900, via release mechanisms not shown here. Alternative embodiments for instrument attachment, not shown here, may also include recesses within the pulley attachment portion 970, with a key-type profile, that an insertion instrument tip inserts into, to manipulate the graft pulley 900.

Graft pulley 900 is preferably inserted into bone tunnel 416 until reaching the bone tunnel blind end 431. At this position, the graft pulley 900 may mechanically fix or anchor with the blind end 431 using a variety of mechanical anchoring or fixing embodiments. A threaded tip or self tapping tip 910 is shown, which operates to cut a path into the bone as the threaded tip 910 is rotated and advanced, through the application of force and torque on the graft pulley 900. Tip 910 may also include a drill bit portion (not shown here) that acts so as to drill a small pilot hole in advance of the threaded tip. Alternatively, a pilot hole could be drilled ahead of time using another instrument. Graft pulley 900 need only tap into the bone to an extent to provide minimal fixation, sufficient only to position a ligament graft 550 as described earlier and not come loose or fall out chronically after the procedure. Alternative embodiments may include bone anchors that tap further into the bone tunnel blind end to provide stronger fixation and potentially permanent ligament positioning without the need for additional fixation elements or components. In general, the expected load on pulley 900 for insertion and positioning of the ligament graft 550 may be relatively small, therefore the thread pitch, size and thread count of graft pulley 900 may be comparably less than existing suture anchors that are known. Following the positioning of the ligament 550, the ligament 550 may be held in place with minimal tension, as a suitable fixation device such as a cannulated screw or expandable device, may be inserted within the bone tunnel to secure the ligament 550 therein. Alternatively, following positioning of the ligament graft 550, one suture end 531 may be withdrawn, so as to fully remove the suture 130 from the tunnel.

In one embodiment, the anchor portion or tip 910 is operable to fully embed into the bone tunnel, as shown in FIG. 9. This allows the graft pulley 900 to take up minimal space within the tunnel, leaving the maximum length of tunnel for the ligament fixation. This may potentially allow the bone tunnel to be drilled to a shorter depth 432 such that blind end 431 is substantially spaced from the distal end of the femur capsule 418, thereby causing less trauma to the patient and improving procedural outcomes.

Graft pulley 900 may be inserted using an instrument similar in spirit to the placement instrument described earlier. Alternative embodiments of placement instruments, suture management and method of placing an anchor may also be described in ArthroCare application Ser. No. 11/140,237, filed May 26, 2005, entitled “Threaded Knotless Suture Anchoring Device and Method,” U.S. application Ser. No. 12/487,338, filed Jun. 18, 2009, entitled “Independent Suture Tensioning and Snaring Apparatus” and U.S. application Ser. No. 12/487,389, filed Jun. 18, 2009, entitled “Independent Suture Tensioning and Snaring Apparatus,” all of which are herein incorporated by reference.

Graft pulley 900 may also be used in alternative target areas such as the shoulder. For example, pulley 900 may be used to attach the rotator cuff to the humeral head during a procedure to repair a rotator cuff. Pulley 900 may be used in any target tissue where a suture may be used to attach prosthesis, ligament or other soft tissue to bone tissue, such as a biceps tendon reattachment procedure.

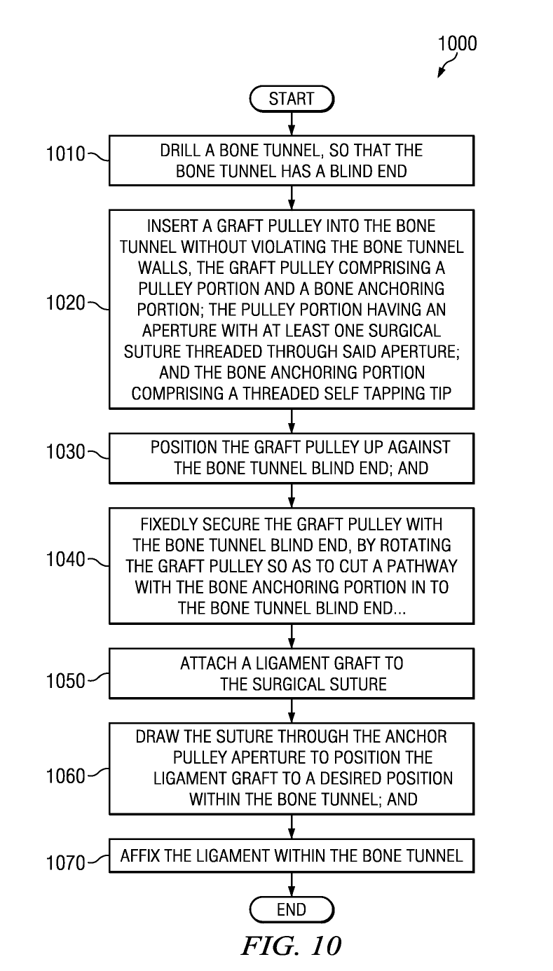

FIG. 10 describes a medical procedure to place a graft pulley within a bone tunnel located in a patient's knee, including the steps of: drilling a bone tunnel, so that the bone tunnel has a blind end (1010) the blind end spaced substantially from a femur distal end capsule, followed by inserting a graft pulley into the bone tunnel (1020). The graft pulley is sized so as to be inserted into the tunnel without violating the bone tunnel walls. This graft pulley comprises a pulley portion and a bone anchoring portion. The pulley portion includes an aperture with at least one surgical suture threaded through said aperture and the bone anchoring portion is operable to fix the graft anchor with the bone tunnel blind end, such as a self tapping tip. The pulley is then placed up against the tunnel blind end (1030) and then rotated and advanced beyond the tunnel blind end (1040), so that the pulley portion is disposed substantially within the bone tunnel blind end, to fixedly secure the graft pulley within the bone tunnel blind end. The medical procedure may also include the steps of attaching a ligament graft to the surgical suture (1050), drawing the suture through the anchor pulley aperture to position the ligament graft to a desired position within the bone tunnel (1060) and affixing the ligament within the bone tunnel (1070).

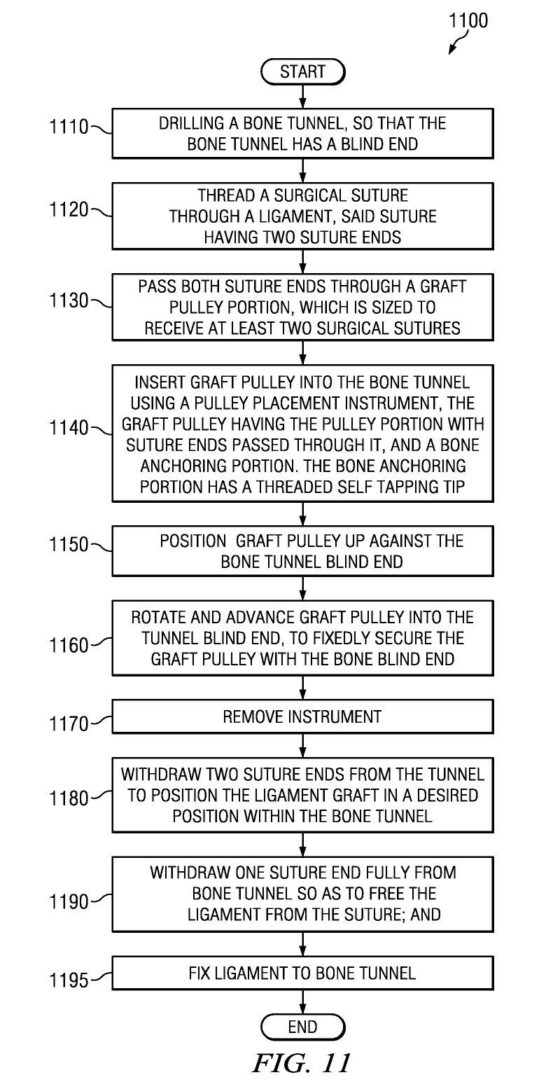

FIG. 11 describes a medical procedure to place a graft pulley within a bone tunnel, including the steps of: drilling a bone tunnel, so that the bone tunnel has a blind end spaced substantially from the distal end of a bone capsule, such as a femur or scapula capsule (1110) followed by threading a surgical suture though a ligament, said suture having two suture ends (1120). Both ends are then passed through a graft pulley, pulley portion, which is sized and shaped to receive at least two surgical sutures (1130). The graft pulley is then inserted into the bone tunnel using a pulley placement instrument (1140), the graft pulley having pulley portion with the suture ends passed through it, and a bone anchoring portion. The bone anchoring portion has a threaded self tapping tip. The graft pulley is then positioned up against the bone tunnel blind end (1150) and then rotated and advanced into the tunnel blind end, so as that the anchoring portion is disposed substantially within the blind end, to fixedly secure the graft pulley within the bone blind end (1160), sufficient to position a ligament into position, but not to permanently hold the ligament in place. The instrument may then be removed (1170), and the two suture ends withdrawn together from the tunnel to position the ligament graft into a desired position within the bone tunnel (1180). Once the ligament is in position, one suture end may then be fully withdraw from the bone tunnel (1190) so as to free the ligament from the suture before affixing the ligament to within the bone tunnel (1195).

Although only a few embodiments of the present invention have been described, it should be understood that the present invention may be embodied in many other specific forms without departing from the spirit or the scope of the present invention. Therefore, the present examples are to be considered as illustrative and not restrictive, and the invention is not to be limited to the details given herein, but may be modified within the scope of the appended claims.

What is claimed is:

1. A graft pulley for positioning a ligament graft within a selected bone tunnel comprising:
a pulley portion having an aperture, the aperture sized to receive at least one surgical suture; and
a bone anchoring portion operable to selectively advance into a bone tunnel blind end, so as to be disposed within the bone tunnel blind end and fixedly secure the graft pulley with the bone; and
wherein the graft pulley is sized to not violate the bone tunnel walls.

2. The graft pulley of claim 1 wherein the graft anchoring portion further comprises a self tapping tip, operable to cut a pathway into the bone tunnel blind end as the anchoring portion is advanced.

3. The graft pulley of claim 1 wherein the graft anchoring portion comprises a shaft with a threaded portion, operable to self tap the anchor portion into the bone tunnel blind end.

4. The graft pulley of claim 1 wherein the bone tunnel has a first cross section dimension and the graft pulley has a second cross section dimension and wherein the first cross section dimension is greater than the second cross section dimension, so that the graft pulley does not engage the tunnel walls.

5. The graft pulley of claim 4, wherein the first cross section is at least 0.5 mm greater than the second cross sectional dimension.

6. The graft pulley of claim 1 further comprising an attachment portion adapted to removably connect the graft pulley with a graft pulley placement instrument.

7. The placement assembly of claim 6 wherein the graft pulley placement instrument comprises a handle with a suture securing portion, adapted to manage the surgical suture.

8. The graft pulley of claim 1 wherein a portion of the graft pulley comprises a radiopaque material.

9. A tissue pulley for positioning a target tissue proximal to a selected bone tunnel comprising:
a pulley portion having an aperture, the aperture sized to receive at least one surgical suture; and
a bone anchoring portion, comprising a threaded surface, operable to selectively advance the tissue pulley beyond a bone tunnel blind end, so as to be disposed within the bone tunnel blind end to fixedly secure the tissue pulley with a bone tunnel; and
wherein the tissue pulley is sized so as not to engage the bone tunnel walls.

10. The tissue pulley as in claim 9 wherein the tissue is a rotator cuff and the bone is a humeral head.

11. The graft pulley of claim 9, the tissue pulley having a cross sectional dimension and the bone tunnel having a diameter, the cross sectional dimension being at least 0.5 mm less than the tunnel diameter.

12. A method for placing a graft pulley into a bone tunnel blind end within a patient's knee comprising:
drilling a bone tunnel, so that the bone tunnel has a blind end and tunnel walls, the blind end substantially spaced from a femur capsule distal end;
inserting a graft pulley into the bone tunnel, wherein the graft pulley is sized so to not violate the bone tunnel walls during insertion, and the graft pulley comprises a pulley portion and a bone anchoring portion;
the pulley portion having an aperture with at least one surgical suture passed through the aperture; and
the bone anchoring portion comprising a threaded self tapping tip;
positioning the graft pulley anchoring portion up against the bone tunnel blind end; and
rotating the graft pulley so that the anchoring portion advances into the tunnel blind end, so as to be disposed within the bone tunnel blind end to fixedly secure the graft pulley with the bone.

13. The method of claim 12 further comprising:
attaching a ligament graft to the surgical suture;
drawing the suture through the anchor pulley aperture to position the ligament graft to a desired position within the bone tunnel; and
affixing the ligament within the bone tunnel.

14. A method for placing a graft pulley within a bone tunnel blind end comprising:
drilling a bone tunnel, so that the bone tunnel has a blind end, the blind end spaced substantially from a bone capsule distal end;
threading a surgical suture though a ligament, said suture having two suture ends;
passing the two suture ends through a graft pulley aperture, wherein the aperture is sized to receive at least two surgical sutures;
inserting a graft pulley into the bone tunnel using a pulley placement instrument, the graft pulley sized so as not to engage the tunnel walls and further comprising a bone anchoring portion having a threaded self tapping tip;
positioning the bone anchoring portion up against the bone tunnel blind end; and
rotating and advancing the graft pulley into the tunnel blind end, so that a portion of the bone anchoring portion is disposed within the bone tunnel blind end, to fixedly secure the graft pulley within the bone blind end.

15. The method of claim 14 further comprising:
removing the pulley placement instrument;
drawing the two suture ends out of the tunnel to position the ligament graft in a desired position within the bone tunnel;
withdrawing one suture end to withdraw suture from bone tunnel; and
affixing ligament within bone tunnel.
